(19) United States
(12) Patent Application Publication
(10) Pub. No.: US 2019/0117124 A1
(43) Pub. Date: Apr. 25, 2019

(54) SENSOR PAD FOR MONITORING USER POSTURE

(71) Applicant: MedicusTek, Inc., Taipei (TW)

(72) Inventors: Chia-Ming Hsu, Taipei (TW); Yu-Chun Hsu, Taipei (TW); Yi-Yuan Chen, Taipei (TW)

(73) Assignee: MedicusTek, Inc., Taipei (TW)

(21) Appl. No.: 15/788,451

(22) Filed: Oct. 19, 2017

(51) Int. Cl. A61B 5/11; A61B 5/00

(52) U.S. Cl. CPC A61B 5/1116; A61B 5/742; A61B 5/1126; A61B 5/6891; A61B 5/0015; A61B 5/6802; A61B 5/6892; A61B 5/7278

(57) ABSTRACT

A sensor pad for monitoring user posture. The sensor pad includes a sensor array having first layer electrodes disposed on a first substrate that faces a second substrate, second layer electrodes disposed on a second substrate that faces the first substrate, a spacer layer disposed between the first substrate and the second substrate and having holes each corresponding to an overlapping region of the first layer electrodes and the second layer electrodes, where the first layer electrodes are configured to contact the second layer electrodes through at least one of the holes via a corresponding overlapping region when an external force is applied on the sensor pad by a user, a sensing circuit configured to generate an output signal in response to the external force, and a sensor correction circuit configured to prevent the sensing circuit from generating a false positive signal in the output signal.

### BACKGROUND

A long term analysis of user posture and changes in the posture while lying in the bed may facilitate the detection of underlying health conditions and reactions to prescribed medications. For example, the changes in the posture may correspond to a breathing rhythm, a frequency of cough, and other actions of a patient. In particular, such changes may inform a medical practitioner regarding the patient's sleep patterns, amount of activity, body movements reflecting physical pain, and the susceptibility to bedsore due to lack of movement. Understanding these non-verbal signals produced by the patient's body may be particularly beneficial to patients who have difficulties communicating with the health care professionals and nursing staff.

### SUMMARY

In general, in one aspect, the invention relates to a sensor pad for monitoring user posture. The sensor pad includes a sensor array that includes a plurality of first layer electrodes disposed on a first substrate that faces a second substrate, a plurality of second layer electrodes disposed on a second substrate that faces the first substrate, a spacer layer disposed between the first substrate and the second substrate and comprising a plurality of holes each corresponding to an overlapping region of the plurality of first layer electrodes and the plurality of second layer electrodes, wherein the plurality of first layer electrodes are configured to contact the plurality of second layer electrodes through at least one of the plurality of holes via a corresponding overlapping region when an external force is applied on the sensor pad by a user, a sensing circuit configured to generate an output signal in response to the external force, and a sensor correction circuit configured to prevent the sensing circuit from generating a false positive signal in the output signal.

In general, in one aspect, the invention relates to a system for monitoring user posture. The system includes (i) a display configured to display posture monitoring data of a user, and (ii) a sensor array including a plurality of first layer electrodes disposed on a first substrate that faces a second substrate, a plurality of second layer electrodes disposed on a second substrate that faces the first substrate, a spacer layer disposed between the first substrate and the second substrate and comprising a plurality of holes each corresponding to an overlapping region of the plurality of first layer electrodes and the plurality of second layer electrodes, wherein the plurality of first layer electrodes are configured to contact the plurality of second layer electrodes through at least one of the plurality of holes via a corresponding overlapping region when an external force is applied on the padding by the user, a sensing circuit configured to generate an output signal in response to the external force, and a sensor correction circuit configured to prevent the sensing circuit from generating a false positive signal in the output signal, wherein the display posture monitoring data is based at least on the output signal.

In general, in one aspect, the invention relates to a method for monitoring user posture. The method includes (i) disposing a sensor array under a user, the sensor array including a plurality of first layer electrodes disposed on a first substrate that faces a second substrate, a plurality of second layer electrodes disposed on a second substrate that faces the first substrate, and a spacer layer disposed between the first substrate and the second substrate and comprising a plurality of holes each corresponding to an overlapping region of the plurality of first layer electrodes and the plurality of second layer electrodes, wherein the plurality of first layer electrodes are configured to contact the plurality of second layer electrodes through at least one of the plurality of holes via a corresponding overlapping region when an external force is applied on the sensor array by a user, (ii) obtain, using a sensing circuit of the sensor array, an output signal in response to the external force, and (iii) display, based on the output signal, posture monitoring data of the user.

Other aspects and advantages of the invention will be apparent from the following description and the appended claims.

### BRIEF DESCRIPTION OF DRAWINGS

FIG. 1 shows a posture monitoring system according to one or more embodiments of the invention.

FIG. 2 shows a sensor pad according to one or more embodiments of the invention.

FIG. 3A shows example details of a sensor array of the sensor pad according to one or more embodiments of the invention.

FIG. 3B shows an example cross-sectional view of the sensor pad according to one or more embodiments of the invention.

FIG. 3C shows an example perspective view of the sensor pad according to one or more embodiments of the invention.

FIG. 4 shows an example graph of topographical data according to more or more embodiments of the present disclosure.

FIG. 5 shows a method flow chart according to one or more embodiments of the invention.

FIG. 6A shows an example circuit diagram of the sensor array according to one or more embodiments of the invention.

FIG. 6B shows an example circuit diagram of the sensor array with false positive correction according to one or more embodiments of the invention.

FIG. 6C shows an example circuit diagram of the posture monitoring system according to one or more embodiments of the invention.

FIGS. 7A and 7B show a computing system according to one or more embodiments of the invention.

### DETAILED DESCRIPTION

Specific embodiments will now be described in detail with reference to the accompanying figures. Like elements in the various figures are denoted by like reference numerals for consistency. Like elements may not be labeled in all figures for the sake of simplicity.

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers does not imply or create a particular ordering of the elements nor limit any element to being only a single element unless expressly disclosed, such as by the use of the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a horizontal beam” includes reference to one or more of such beams.

Terms like “approximately,” “substantially,” “nominally,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Although multiple dependent claims are not introduced, it would be apparent to one of ordinary skill in that that the subject matter of the dependent claims of one or more embodiments may be combined with other dependent claims. For example, even though claim 3 does not directly depend from claim 2, even if claim 2 were incorporated into independent claim 1, claim 3 is still able to be combined with independent claim 1 that would now recite the subject matter of dependent claim 2.

In general, embodiments of the invention relate to a posture monitoring system including a sensor pad and a display. A posture is the relative disposition of various parts of a user. The sensor pad includes a sensor array and padding that is disposed between the sensor array and the user. The sensor array produces a signal output when a user applies an external force onto the surface of the sensor pad, in particular the padding. The sensor pad is coupled to a computing device that produces topographical data of the surface of the sensor pad, in particular the padding, by processing the signal output from the sensor array. The topographical data represents a distribution of the user's weight on the surface of the sensor pad, in particular the padding, to determine the user's posture and movement on the sensor pad. In one or more embodiments, the topographical data and the 2-dimensional (2D) geometry of the sensor array are collectively referred to as the three dimensions of the sensor array. In other words, the 2D geometry may correspond to the X-dimension and Y-dimension of the sensor array while the topographical data corresponds to the Z-dimension of the sensor array.

In one or more embodiments, the sensor array includes a first set of electrode layers disposed in parallel on a side of a first substrate that faces a second substrate, and a second set of electrode layers disposed in parallel on a side of the second substrate that faces the first substrate. The second set of electrode layers intersect (e.g., being perpendicular with respect to) the first set of electrode layers to form a two-dimensional matrix grid. The first set of electrode layers is in electrical contact with the second set of electrode layers when a user applies an external force on the surface of the sensor pad. The padding transfers the external force into contact pressure where the first set of electrode layers is in electrical contact with the second set of electrode layers. The contact pressure causes a change in contact resistance that produces the output signal.

In general, the phrase of “in electrical contact with”, as used above, is defined as two objects that are in physical contact and able to transfer electrons between each other. For example, when an electrode layer from the first set of electrode layers is in physical contact with another electrode layer from the second set of electrode layers, the two electrode layers are able to transfer electrons between each other.

In one or more embodiments, the sensor array may produce a correct signal output when a user applies an external force onto the surface of the sensor pad. The sensor array may also produce an incorrect signal output, e.g., due to a ghost current, when a user applies an external force onto the surface of the sensor pad. The incorrect signal output is an output signal that contains false positive data. A false positive data is a data set that includes one or more subsets of data that depict an area of external force on the sensor pad where the user did not apply any external force.

In one or more embodiments, the sensor pad includes a sensor correction circuit that prevents the sensor pad from generating false positive data in the output signal. The sensor correction circuit is controlled by the computing device to provide an input voltage to one of the electrodes of the first set of electrode layers and an additional voltage signal to a predetermined number of electrode layers of the second set of electrode layers. The input voltage and the additional voltage signal are collectively referred to as the de-ghosting signal.

One or more embodiments of the sensor pad may be deployed in healthcare facilities (e.g. hospitals, clinics, nursing homes, etc.) based on different padding material to be included in a mattress, a seat cushion, a shoe pad, and a wearable article worn by the user.

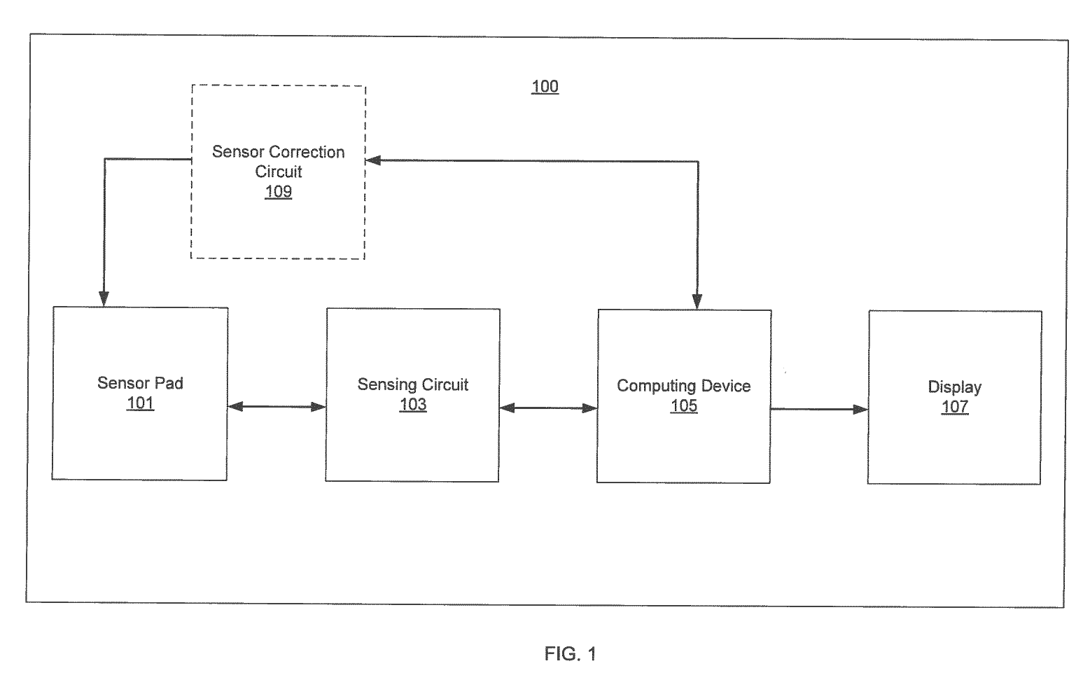

In accordance with one or more embodiments of the invention, FIG. 1 illustrates a posture monitoring system (100). In one or more embodiments, one or more of the modules and elements shown in FIG. 1 may be omitted, repeated, and/or substituted. Accordingly, embodiments of sensor pad for monitoring user posture should not be considered limited to the specific arrangements of modules shown in FIG. 1.

As shown in FIG. 1, the posture monitoring system (100) includes a sensor pad (101), a sensing circuit (103), a computing device (105), a display (107), and a sensor correction circuit (109). The various components and structures of the posture monitoring system (100) listed above may interact directly or indirectly with one another. Each of these components will be described below in more detail.

In one or more embodiments, the sensor pad (101) may be a pressure sensing mat. The sensing mat may be a square or rectangular flexible pressure sensing mat that includes a six-by-six electrode array matrix. In one or more embodiments, the particular shape, size, etc., of the sensor pad (101) and the size of the electrode array matrix in the sensor pad (101) may vary, based on the particular application of the sensor pad (101), without departing from the scope of the invention.

In one or more embodiments, the computing device (105) includes one or more hardware and/or software components. For example, the computing device (105) may include one or more computer processors, non-persistent storage (e.g., volatile memory, such as RAM, cache memory), persistent storage (e.g., a hard disk, an optical drive such as a compact disk (CD) drive or digital versatile disk (DVD) drive, a flash memory, etc.), a communication interface (e.g., Bluetooth interface, infrared interface, network interface, optical interface, etc.), and numerous other elements and functionalities.

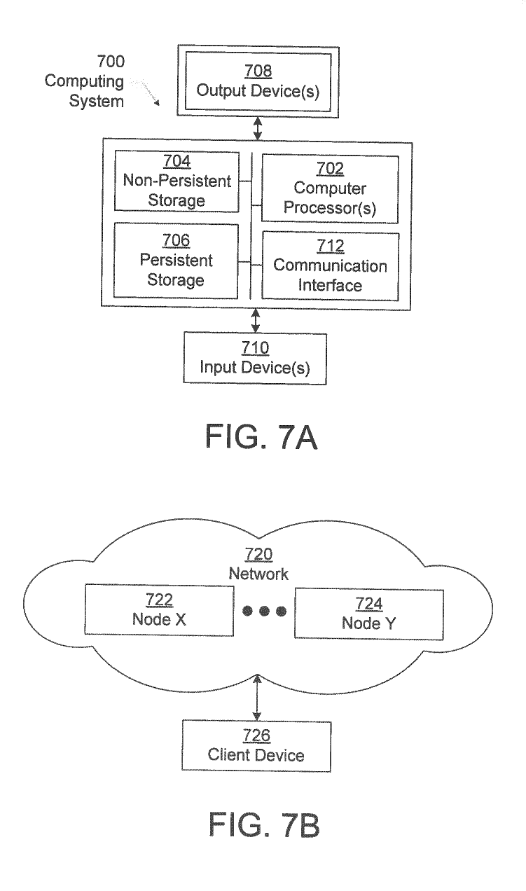

In one or more embodiments, the display (107) may be one or more output devices, such as a screen (e.g., a liquid crystal display (LCD), a plasma display, touchscreen, cathode ray tube (CRT) monitor, projector, or other display device), a printer, external storage, or any other output device. In one or more embodiments, the computing device (105) and display (107) correspond to the computing system, or a portion thereof, described in reference to FIG. 7 below.

In one or more embodiments, the sensing circuit (103) may include one or more analog and/or digital circuitry configured to generate an output signal in response to an external force applied by a user to the sensor pad (101). In one or more embodiments, the sensing circuit (103) transfers the output signal received from the sensor pad (101) to the computing device (105) and receives commands from the computing device (105). The computing device (105) processes the output signal received from the sensor pad (101) and generates a corresponding value for sending to the display (107).

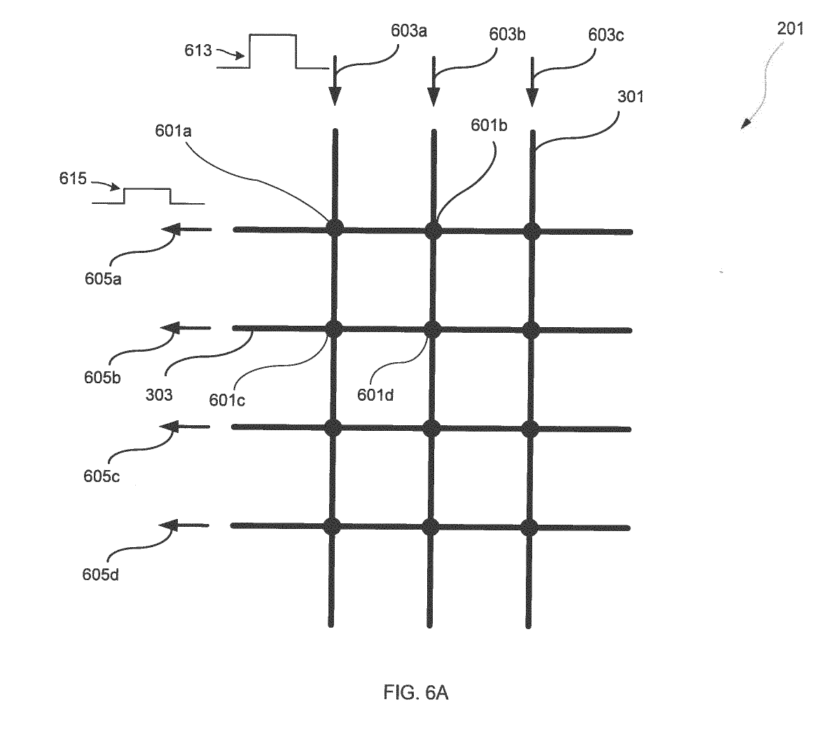

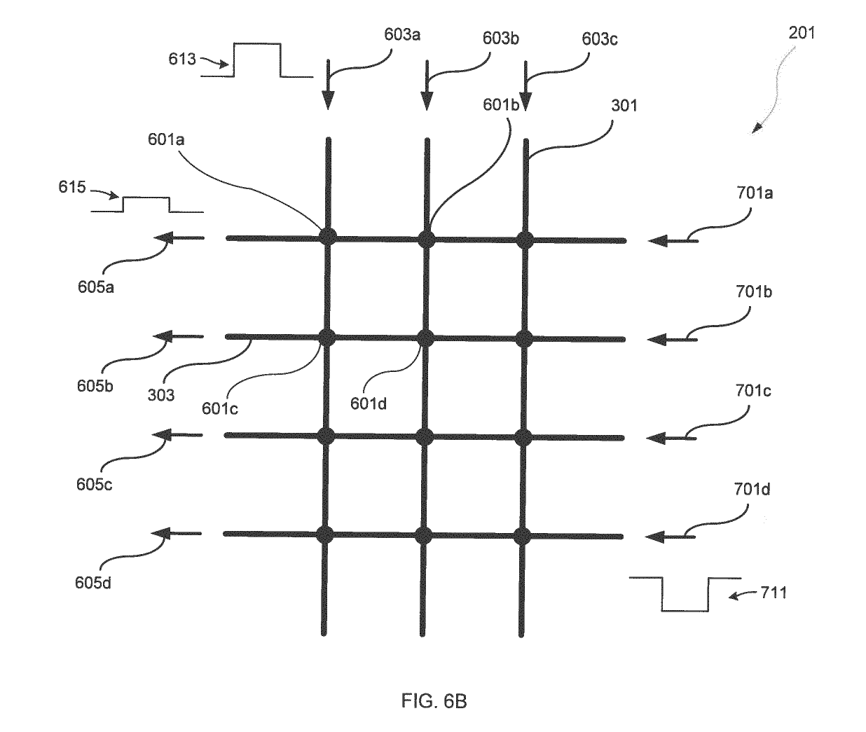

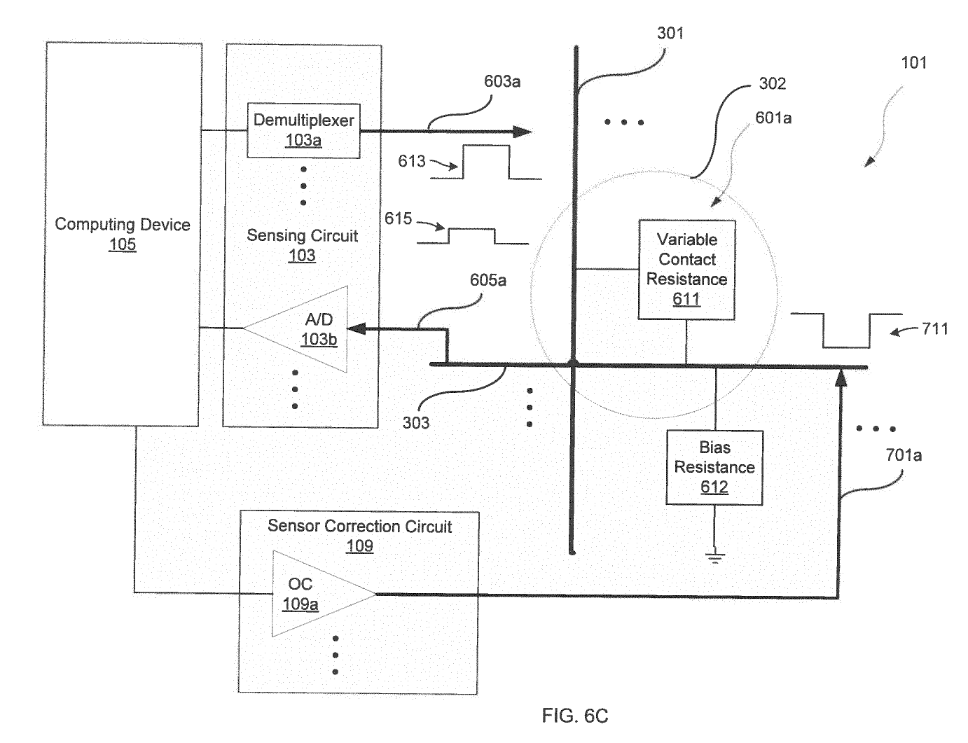

In one or more embodiments, the sensor correction circuit (109) may include one or more analog and/or digital circuitry configured to prevent the sensing circuit (103) from generating a false positive signal in the output signal. Further details of the sensing circuit (103) and the sensor correction circuit (109) are described in reference to FIGS. 6A-6C below. In one or more embodiments, the sensing circuit (103) and/or the sensor correction circuit (109) are combined with the sensor pad (101). In one or more embodiments, the sensing circuit (103) and/or the sensor correction circuit (109) are combined with the computing device (105). In one or more embodiments, the sensing circuit (103) and/or the sensor correction circuit (109) are separate from the sensor pad (101) and the computing device (105).

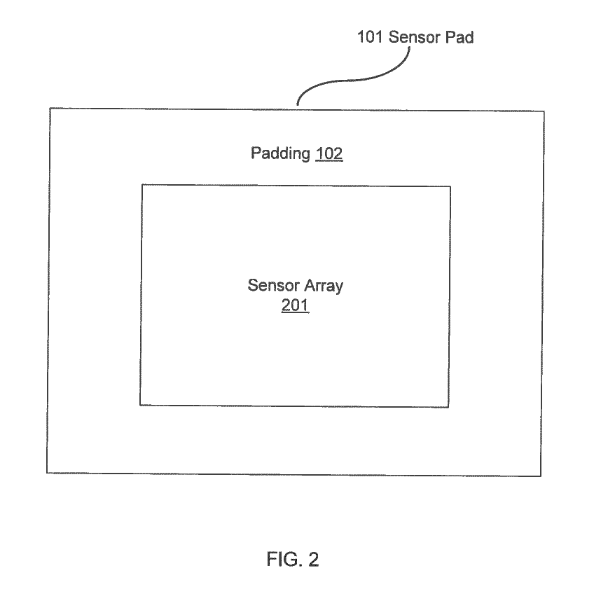

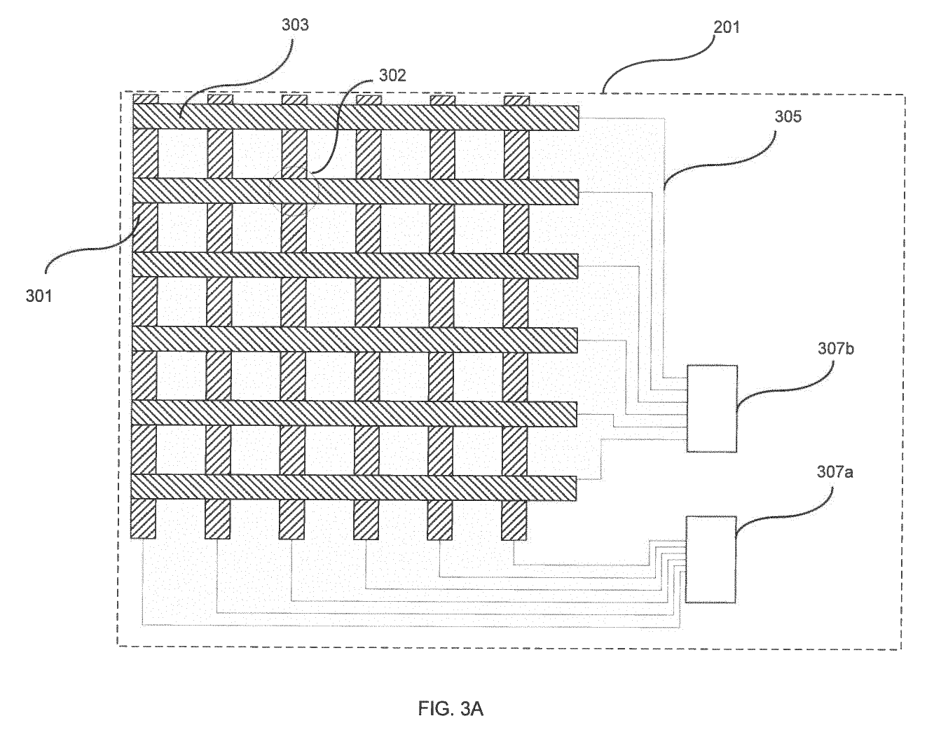

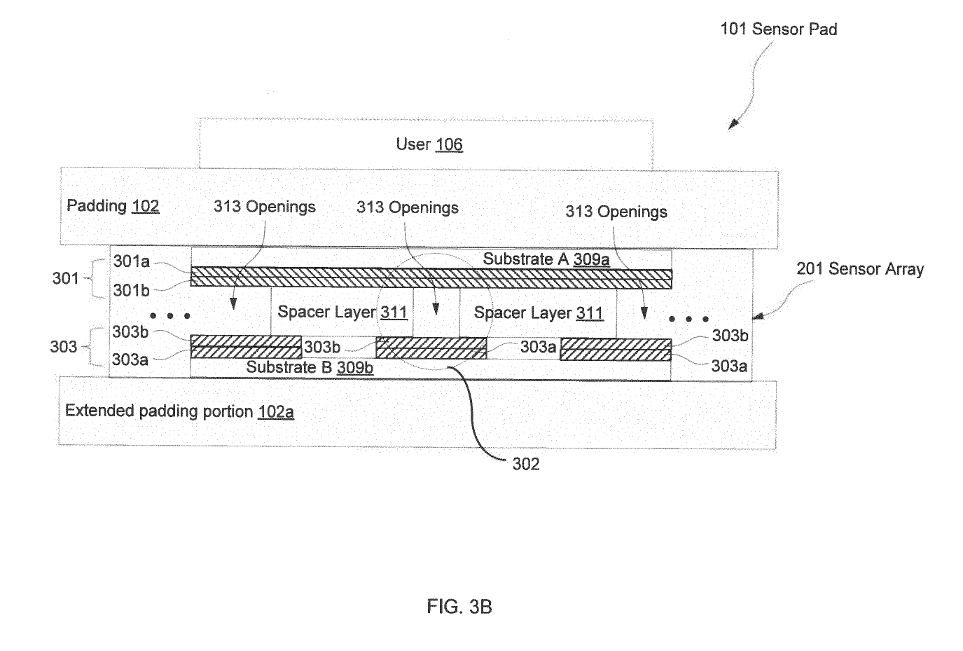

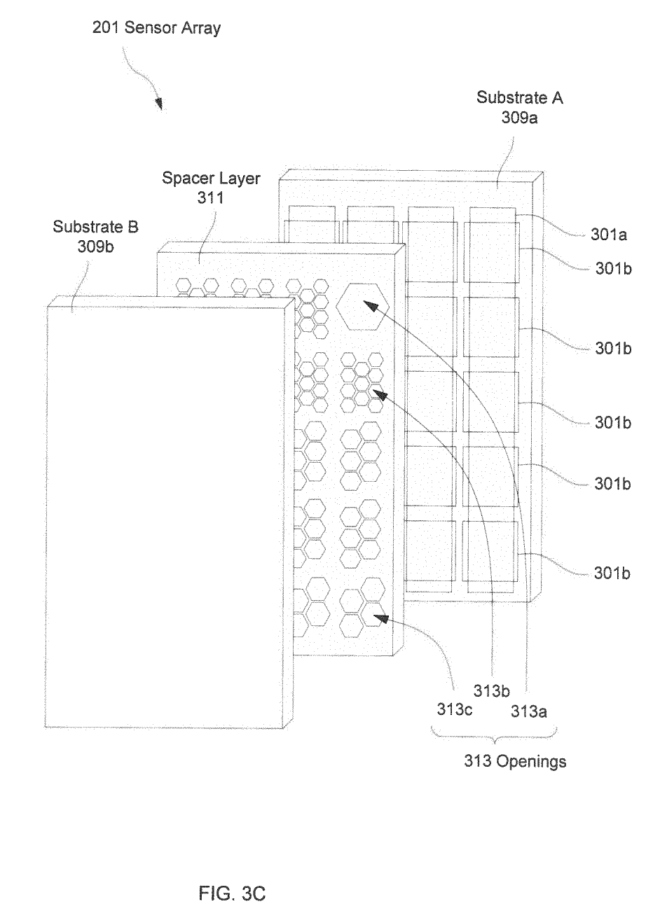

In one or more embodiments, the sensor pad (101) may include pressure sensors disposed on a mattress, a seat cushion, a shoe pad, and a wearable article worn by the user. Each sensor is referred to as a sensing point of the sensor pad (101). The sensor pad (101) is connected to the computing device (105), via the sensing circuit (103) and the sensor correction circuit (109), to receive input signals from the computing device (105) and to generate an output signal to the computing device (105) that is a function of external force applied to a surface of the sensor pad (101). In one or more embodiments, the input signal corresponds to a voltage to be applied to the pressure sensors of the sensor pad (101) and the output signal corresponds to a sensed current received from the pressure sensors of the sensor pad (101). For example, the sensed current may be derived from a resistive material of the pressure sensor in response to the applied voltage. Further details of the sensor pad (101) are described in reference to FIGS. 2 and 3A-3C below.

In accordance with one or more embodiments, FIG. 2 shows a sensor pad (101) of the posture monitoring system (100). In one or more embodiments, one or more of the modules and elements shown in FIG. 2 may be omitted, repeated, and/or substituted. Accordingly, embodiments of sensor pad for monitoring user posture should not be considered limited to the specific arrangements of modules shown in FIG. 2.

As shown in FIG. 2, one or more embodiments of the sensor pad (101) include a sensor array (201) disposed about at least a portion of a padding (102). In one or more embodiments, the padding (102) is constructed using cloth or some other soft material, such as cotton, flax wool, ramie, silk, leather and fur etc. The padding (102) may be integrated with or as a part of a mattress, a seat cushion, a shoe pad, or a wearable article worn by the user. In one or more embodiments, the padding (102) includes one or more hollow portions that is inflatable with air, such as in an air mattress. The sensor array (201) may be a square or rectangular flexible six-by-six electrode array matrix. For example, the sensor array (201) may be inserted in a mattress, a seat cushion, a shoe pad, or a wearable article worn by the user where the padding (102) is included. In another example, the sensor array (201) may be inserted in the padding (102) that is separate from and disposed over a mattress, a seat cushion, a shoe pad, or a wearable article worn by the user. In one or more embodiments, the particular shape and size of the sensor array (201) may vary, based on the particular application of the sensor array (201), without departing from the scope of the invention.

In accordance with one or more embodiments, FIG. 3A shows an example details of the components within the sensor array (201). In one or more embodiments, one or more of the modules and elements shown in FIG. 3A may be omitted, repeated, and/or substituted. Accordingly, embodiments of sensor pad for monitoring user posture should not be considered limited to the specific arrangements of modules shown in FIG. 3A.

As shown in FIG. 3A, the sensor array (201) includes a first layer electrode (301), a second layer electrode (303), wires (305), and connectors (307a-b). The various components and structures of the sensor array (201) listed above may interact directly or indirectly with one another. Each of these components will be described below in more detail.

In one or more embodiments, the first layer electrode (301) may be a conducting electrode in the shape of a rectangle. In one or more embodiments, the sensor array (201) may include more than one first layer electrode (301). In the case where the sensor array (201) includes more than one first layer electrodes (301), multiple first layer electrodes (301) are disposed in parallel to each other forming a 2D plane, and are highlighted in FIG. 3A using the same hatch pattern. In one or more embodiments, multiple first layer electrodes (301) are constructed from depositing electrically conducting material onto a substrate to form the pattern of parallel rectangles shown in FIG. 3A. As used herein, electrically conducting material is a material that allows the flow of electrical current in one or more directions.

In one or more embodiments, the particular shape and size of the first layer electrode (301), the number of first layer electrodes (301) in the sensor array (201), and the distance between each of the multiple first layer electrodes (301) may vary, based on the particular application of the sensor array (201), without departing from the scope of the invention. For example, the first layer electrode (301) may have a circular, elliptical, or other curvilinear shape.

In one or more embodiments, the second layer electrode (303) may be a conducting electrode in the shape of a rectangle. In one or more embodiments, the sensor array (201) may include more than one second layer electrode (303). In the case where the sensor array (201) includes more than one second layer electrodes (303), multiple second layer electrodes (303) are disposed in parallel to each other forming a 2D plane, and are highlighted in FIG. 3A using the same hatch pattern. In one or more embodiments, the multiple second layer electrodes (303) are constructed from depositing electrically conducting material onto a substrate to form the pattern of parallel rectangles shown in FIG. 3A.

In one or more embodiments, the particular shape and size of the second layer electrode (303), the number of second layer electrodes (303) in the sensor array (201), and the distance between each of the plurality of second layer electrodes (303) may vary, based on the particular application of the sensor array (201), without departing from the scope of the invention. For example, the second layer electrode (303) may have a circular, elliptical, or other curvilinear shape.

In one or more embodiments, the first layer electrode (301) and the second layer electrode (303) are disposed to be perpendicular with each other and overlapping each other in the 2D plane. As shown in FIG. 3A, the first layer electrode (301) is obscured by the second layer electrode (303) in each overlapping region (e.g., overlapping region (302)). In one or more embodiments, the first layer electrode (301) and the second layer electrode (303) may form any angle other than being perpendicular with each other. The first layer electrode (301) and the second layer electrode (303) may have, but is not limited to, the same material composition.

In one or more embodiments, the wires (305) may be electrical wires made of copper (e.g., etched copper pattern in a flexible printed circuit board (flex-PCB) construction) or silver (e.g., silver paste screen-printed onto a laminated polymer film). The wires (305) may be insulated with a plastic material, such as polyethylene terephthalate (PET), thermoplastic polyurethane (TPU), polyimide (PI), etc. The wires (305) may be wires of a ribbon connector with one end of the ribbon connector including one of the connectors (307a-b) and the other end with trimmed and exposed wires (305).

The connectors (307a-b) may be connector heads of a ribbon connector. The connectors (307a-b) may be male or female connector heads. One of the connectors (307a-b) is an input connector and the other of the connectors (307a-b) is an output connector. The connectors (307a-b) are configured to be connected to the I/O circuits of the computing device (105).

In one or more embodiments, although FIG. 3A only shows two connectors (307a-b), the sensor pad (101) may have more than two connectors (307a-b) without departing from the scope of the invention.

As shown in FIG. 3A, in one or more embodiments, the wires (305) electrically connect the connector (307a) with the first layer electrode (301) and the connector (307b) with the second layer electrode (303). One of ordinary skill would appreciate that the connection scheme between the electrode layers and the connectors (307a-b) may vary without departing from the scope of the invention

In accordance with one or more embodiments, FIG. 3B shows an example cross-sectional view of the sensor pad (101) depicted in FIG. 2 and FIG. 3A above. In one or more embodiments, one or more of the modules and elements shown in FIG. 3B may be omitted, repeated, and/or substituted. Accordingly, embodiments of sensor pad for monitoring user posture should not be considered limited to the specific arrangements of modules shown in FIG. 3B.

As shown in FIG. 3B, a user (106) (e.g., a patient, such as a human patient or an animal patient) may be lying down on, sitting on, or stepping on the sensor pad (101). As shown in the cross-sectional view, the sensor array (201) includes a substrate A (309a), a substrate B (309b), a spacer layer (311), and openings (313). The various components and structures of the sensor array (201) listed above may interact directly or indirectly with one another. Each of these components will be described below in more detail.

In one or more embodiments of the invention, the substrate A (309a) and the substrate B (309b) include flexible films, the materials of which may be one independently selected from the group consisting of: polyethylene terephthalate (PET), thermoplastic polyurethane (TPU), polyimide (PI), other plastics, other flexible materials, and combinations thereof. In one or more embodiments, the substrate A (309a) and/or the substrate B (309b) may be supplemented with a padding (102) of cloth or some other soft material, such as cotton, flax wool, ramie, silk, leather and fur etc. that can minimize or reduce noise as well as enhance comfort level when the user (106) interacts with the substrate A (309a) and/or the substrate B (309b).

In one or more embodiments of the invention, the thickness of the substrate A (309a) and the substrate B (309b) may be, but is not limited to, 0.0014 millimeter (mm) or more. In an example construction (referred to as construction A) of the sensor pad (101), the padding (102) may have a nominal thickness 2 centimeter (cm) separating the user (106) from the sensor array (201), and the substrate A (309a) and/or the substrate B (309b) may include a PET film having nominal thickness 0.025 mm laminated with a PI film having nominal thickness 0.025 mm. In another example construction (referred to as construction B) of the sensor pad (101), the padding (102) may have a nominal thickness grater than 5 cm separating the user (106) from the sensor array (201), and the substrate A (309a) and/or the substrate B (309b) may include a PET film having nominal thickness 0.188 mm laminated with a PI film having nominal thickness 0.025 mm. Although the padding (102) is shown to be disposed between the user (106) and the sensor array (201), in one or more embodiments, the padding (102) may extend to include an extended padding portion (102a) to enclose at least a portion of the sensor array (201). For example, while the as-shown portion of the padding (102) may be disposed between the user (106) and the sensor array (201), the extended padding portion (102a) of the padding (102) may be disposed on a different side of the sensor array (201) that is not in contact with the user (106). Further, the padding (102) and substrate A (309a) may be integrated or otherwise combined into a single item. Similarly, the padding (102a) and substrate B (309b) may be integrated or otherwise combined.

In one or more embodiments of the invention, the spacer layer (311) is a flexible and deformable insulating material, which may be one selected from the group consisting of polyethylene terephthalate (PET), thermoplastic polyurethane (TPU), polyimide (PI), synthetic or natural sponge, foamed plastics, and combinations thereof. In one or more embodiments of the invention, the thickness of the spacer layer (311) may be, but is not limited to, 3 mm or more. In the example construction A of the sensor pad (101), the spacer layer (311) may include a TPU film with nominal thickness 0.02 mm. In the example construction B of the sensor pad (101), the spacer layer (311) may include a TPU film with nominal thickness between 0.2 mm and 0.5 mm.

As shown in FIG. 3B, the first layer electrode (301) includes multiple laminated layers (e.g., layer (301a), layer (301b)) and is disposed on the substrate A (309a). Similarly, the second layer electrode (303) includes multiple laminated layers (e.g., layer (303a), layer (303b)) and is disposed on the substrate B (309b). In one or more embodiments, the first layer electrode may be disposed on the substrate B (309b) and the second layer electrode may be disposed on the substrate A (309a). In one or more embodiments, the layer (301a) and/or layer (303a) are constructed using copper (e.g., etched copper pattern in a flex-PCB construction) or silver (e.g., silver paste screen-printed onto a laminated polymer film). For example, the flex-PCB may include the layer (301a) and the substrate A (309a), or the layer (303a) and the substrate B (309b). In another example, the laminated polymer film (screen-printed using silver paste) may include the layer (301a) and the substrate A (309a), or the layer (303a) and the substrate B (309b). In one or more embodiments, the layer (301a) and the layer (303a) are connected to corresponding wires of the wires (305) depicted in FIG. 3A above.

In one or more embodiments, the layer (301b) and/or layer (303b) are constructed using carbon/graphite paste or other electrically resistive material having higher resistance than the layer (301a) and/or layer (303a). For example, the layer (301b) and/or layer (303b) may be screen-printed or otherwise coated, using carbon/graphite paste or other electrically resistive material, over the flex-PCB or the laminated polymer film (with prior screen-printed silver paste). As used herein, the electrically resistive material is an electrically conductive material with higher electrical resistivity than copper or silver. In one or more embodiments, the screen-printed or coated carbon/graphite of the layer (301b) and/or layer (303b) has a thickness in the range of 5-15 micrometer (μm).

In one or more embodiments, the layer (301a) and layer (301b) may have the same nominal dimensions in both width and length directions. For example, the nominal width may be approximately 1 inch. In one or more embodiments, the layer (301b) is wider (e.g., 1.25 inch) than and overlaps the layer (301a) (e.g., 1 mm) in the width direction. In one or more embodiments, the layer (301b) is separated into sections in the length direction where each section overlaps one opening site of the openings (313) along the layer (301a). An example of the layer (301b) separated into sections is described in reference to FIG. 3C below.

In one or more embodiments, the layer (303a) and layer (303b) may have the same nominal dimensions in both width and length directions. In one or more embodiments, the layer (303b) is wider than and overlaps the layer (303a) in the width direction. In one or more embodiments, the layer (303b) is separated into sections in the length direction where each section overlaps one opening site of the openings (313) along the layer (303a). An example of the layer (303b) separated into sections is described in reference to FIG. 3C below.

In one or more embodiments, the spacer layer (311) includes openings (313). The openings (313) include a number of opening sites where each opening site corresponds to (e.g., aligned with) an overlapping region (e.g., overlapping region (302)) of the substrate A (309a) and the substrate B (309b). When the user (106) applies an external force (e.g., by lying down, sitting, or stepping) on the surface of the sensor pad (101), the spacer layer (311) is deformed such that the first layer electrode (301) and the second layer electrode (303) are brought together in electrical contact with each other through the openings (313) in the spacer layer (311). As noted above, more than one first layer electrode (301) and more than one second layer electrode (303) may exist in the sensor array (201). Depending on the size of the area where the user (106) applies the force onto the sensor pad (101), more than one first layer electrode (301) and more than one second layer electrode (303) may be brought together in electrical contact with each other simultaneously through multiple opening sites of the openings (313) in the spacer layer (311).

In one or more embodiments, each opening site of the openings (313) defines a point of contact (referred to as a sensing point of the sensor array (201)) between the first layer electrode (301) and the second layer electrode (303). The area of electrical contact at each opening site when the force is applied to the sensor pad (101) is referred to as the opening site contact area, or simply contact area. The pressure exerted between the first layer electrode (301) and the second layer electrode (303) at each opening site contact area when the force is applied to the sensor pad (101) is referred to as the opening site contact pressure, or simply contact pressure. The electrical resistance between the first layer electrode (301) and the second layer electrode (303) through each opening site contact area when the force is applied to the sensor pad (101) is referred to as the opening site contact resistance, or simply contact resistance.

In one or more embodiments, the contact resistance is a function of the contact pressure. For example within certain range of the contact pressure (referred to as the linear sensitivity range of contact pressure), higher contact pressure may lower the contact resistance, and lower contact pressure may increase the contact resistance. In contrast, the contact resistance may be substantially independent of the contact pressure when the contact pressure is outside of the linear sensitivity range of contact pressure. For example, the contact resistance may approach infinity when the contact pressure is less than the lower limit of the linear sensitivity range of contact pressure. In another example, the contact resistance may approach a constant when the contact pressure exceeds the upper limit of the linear sensitivity range of contact pressure. The linear sensitivity range of contact pressure, or simply linear sensitivity range, is the range of contact pressure between these lower and upper limits. The ratio of the resulting decrease in the contact resistance to an increase in contact pressure at each opening site within the linear sensitivity range is referred to as the opening site sensing sensitivity, or simply sensing sensitivity.

In one or more embodiments, one or more of the thicknesses, contact areas, and material types of the padding (102), substrate A (309a), substrate B (309b), the first layer electrode (301), the second layer electrode (303), and the spacer layer (311) are selected to enhance the sensing sensitivity. For example, the thickness and material type of the padding (102) may be selected such that throughout a predetermined weight range (e.g., 20 kg-500 kg or 44 lb-1100 lb), the weight of the user (106) is transferred into a contact pressure within the linear sensitivity range. In one or more embodiments, in addition to the functionalities of reducing noise and enhancing comfort level of the user (106), the padding (102) is further configured to transfer, collectively with the substrate A (309a) and the substrate B (309b), the weight of the user (106) into a contact pressure, in each contact area throughout the sensor array (201), that is within the linear sensitivity range.

In the example construction A or example construction B of the sensor pad (101), each opening site of the openings (313) may include one or more holes each shaped as a square, rectangle, circle, dot, cross, etc. For example, each opening site may include a single square/rectangle/circle/dot/cross shaped hole. The contact area and the contact resistance are based on the single square/rectangle/circle/dot/cross shaped hole. In another example, each opening site may include multiple square/rectangle/circle/dot/cross shaped holes. The contact area and the contact resistance are based on the combined area and the combined electrical resistance of these multiple square/rectangle/circle/dot/cross shaped holes. Each hole may have an X-dimension or Y-dimension between 0.1 cm and 12 cm. Further, let L denotes the thickness of the first layer electrode (301) and/or the second layer electrode (303), and let A denotes the area of each hole, the ratio L/A is less than 12,000 in one or more embodiments. Additional example constructions are listed in TABLE 1 below. While TABLE 1 corresponds to a user weight range of 20 Kg-500 Kg, one or more embodiments may correspond to a different user weight range or a subset of the user weight range shown in TABLE 1. For example, an embodiment for a baby as the user may correspond to a user weight range of 1 Kg-10 Kg.

In accordance with one or more embodiments, FIG. 3C shows an example of a 3D perspective view of the sensor array (201) in the sensor pad (101) depicted in FIGS. 2, 3A, and 3B above. In one or more embodiments, one or more of the modules and elements shown in FIG. 3C may be omitted, repeated, and/or substituted. Accordingly, embodiments of sensor pad for monitoring user posture should not be considered limited to the specific arrangements of modules shown in FIG. 3C.

In the example shown in FIG. 3C, the layer (301b) is wider than and overlaps the layer (301a) in the width direction. Further, the layer (301b) is separated into sections in the length direction where each section overlaps one opening site of the openings (313) along the layer (301a). The openings (313) includes an example opening site A (313a) having a single hexagonal shaped hole, an example opening site B (313b) having a matrix of 11 hexagonal shaped holes, an example opening site C (313c) having a matrix of 4 hexagonal shaped holes, etc. The layer (303a) and layer (303b) are obscured by the substrate B (309b) and are not explicitly shown. Although the layer (301b) is shown as separated into sections and the openings (313) are shown as including different layouts of opening sites in the example depicted in FIG. 3C, other configurations of the layer (301b) and the openings (313) are also possible in other examples. For example, the layer (301b) may include a contiguous rectangular shape superimposing multiple opening sites of the openings (313) throughout at least a portion of the entire length of the layer (301b) while the openings (313) may include consistent layouts of opening sites throughout at least a portion of the spacer layer (311).

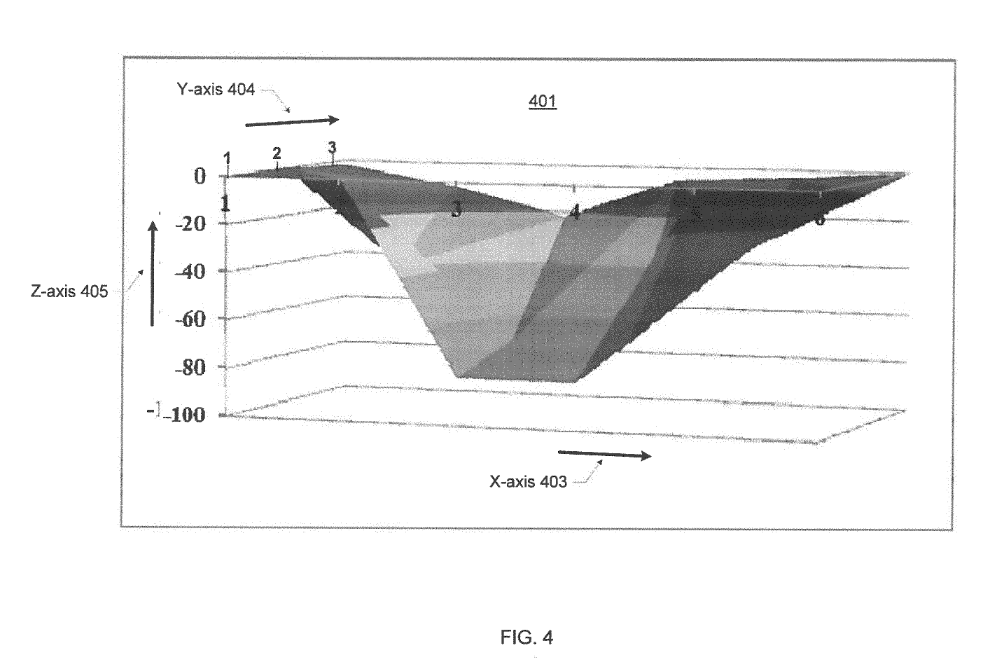

In accordance with one or more embodiments, FIG. 4 shows a graph, which is described below, of a topographical data (401) of the surface of the sensor pad (101). The output signal from the sensor pad (101) generated by the sensor array (201) is processed by the computing device (105) depicted in FIG. 1 above to produce the topographical data (401) of FIG. 4. In one or more embodiments, the output signal is dependent on a contact resistance between the first layer electrode (301) and the second layer electrode (303) at the openings (313) of the spacer layer (311). In one or more embodiments, the topographical data (401) is sent from the computing device (105) to the display (107) for displaying the graph.

TABLE 1

| User weight 20 Kg-500 Kg | Example Mattress Pad | Example Mattress |
|---|---|---|
| Padding thickness (mm) | 0-20 | 0 |
| Substrate thickness (mm) | 0.025-0.3 | 0.025-0.3 |
| Spacer thickness (mm) | 0.02-3 | 0.02-3 |
| Opening contact area (cm²) | 1-96 | 1-96 |
| Contact pressure (g/cm²) | 5-70 | 5-70 |
| Sensitivity (Ohm/cm²⁾ | 100-100000 | 100-100000 |

In accordance with one or more embodiments, FIG. 4 shows a graph, which is described below, of a topographical data (401) of the surface of the sensor pad (101). The output signal from the sensor pad (101) generated by the sensor array (201) is processed by the computing device (105) depicted in FIG. 1 above to produce the topographical data (401) of FIG. 4. In one or more embodiments, the output signal is dependent on a contact resistance between the first layer electrode (301) and the second layer electrode (303) at the openings (313) of the spacer layer (311). In one or more embodiments, the topographical data (401) is sent from the computing device (105) to the display (107) for displaying the graph.

As shown in FIG. 4, the graph of the topographical data (401) contains three major axes: an x-axis (403), a y-axis (404), and a z-axis (405). Each of the three axes will be described below in more detail.

In one or more embodiments, the x-axis (403) and the y-axis (404) represent the 2D planar surface of the sensor array (201). Each point on the X-Y plane represents a point of contact (i.e., a sensing point) between the first layer electrode (301) and the second layer electrode (303). The number of points on each of the x-axis (403) and the y-axis (404) depends on the size of the sensor array (201). For example, in a sensor array (201) with six-by-six electrode layer array matrix, there are six data points on the x-axis (403) and six data points on the y-axis (404).

In one or more embodiments, the z-axis (405) represents a depth value that depicts a distribution of a user's weight on the surface of the sensor pad (101). When a user or a user's weight presses down on the surface of the sensor pad (101) and causes a part of the first layer electrode (301) to contact a part of the second layer electrode (301) through the opening (313) in the spacer layer (311), the physical and electrical contact between the first layer electrode (301) and the second layer electrode (303) generates the output signal that is outputted from the sensor pad (101) to the computing (105). The computing device (105) processes the output signal to calculate a z-axis (405) depth value. For example, the value (i.e., z-axis (405) depth value) may represent a magnitude of a sensed analog current induced by an applied voltage across a contact resistance at a sensing point of the sensor pad (101). In one or more embodiments, the contact resistance decreases as the contact pressure increases at the sensing point and, therefore, the value also represents the contact pressure at the sensing point. In this context, each sensing point is a pressure sensor. For example within a certain range of contact pressure, higher contact pressure may lower the contact resistance with a substantially linear relationship between contact resistance and contact pressure.

As shown in FIG. 4, the topography data (401) is obtained from a six-by-three sensor array corresponding to x=1, 2, 3, 4, 5, 6 along the x-axis (403) and y=1, 2, 3 along the y-axis (404). As shown in FIG. 4, the user or the user's weight is applying a larger external force near the center of the x-axis (403) (corresponding to x=3, 4) and at one side of the y-axis (404) (corresponding to y=1). In other words, based on the topographical data (401) shown in FIG. 4, it is detected that a part of the user's body pressing down on the sensor pad (101) is approximately at the center of one side of the sensor pad (101).

In one or more embodiments, the user's posture and a movement by the user on the surface of the sensor pad (101) are determined using the topographical data (401). From the movement by the user on the surface of the sensor pad (101), healthcare professionals may determine changes in the user's posture, breathing rhythm, the user's frequency of cough, and other actions of the user while the user is on the sensor pad.

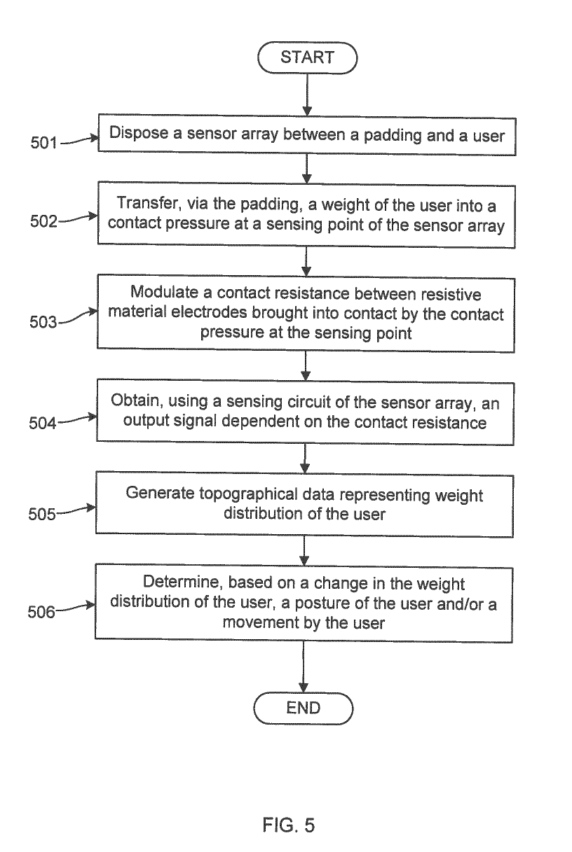

In accordance with one or more embodiments, FIG. 5 shows a flow chart of the general operation of a posture monitoring system. In one or more embodiments, the method as shown in FIG. 5 is a combination of hardware and computer-implemented method. For example, the method depicted in FIG. 5 may be practiced using the posture monitoring system (100) and the sensor pad (101) described in reference to FIGS. 1, 2, 3A, 3B, and 3C above. In one or more embodiments, one or more of the elements shown in FIG. 5 may be omitted, repeated, and/or performed in a different order. Accordingly, embodiments of the sensor pad for monitoring user posture should not be considered limited to the specific arrangements of elements shown in FIG. 5.

Initially in STEP 501, a sensor array is disposed between a padding and a user. In one or more embodiments, the sensor array is based on the description of FIGS. 2 and 3A-3C above. In particular, the sensor array includes multiple sensing points across a 2D planar surface of the padding.

In Step 502, a weight of the user is transferred via the padding into a contact pressure at a sensing point of the sensor array. In one or more embodiments, the contact pressure is maintained by the padding to be within a linear sensitivity range based on the weight of the user.

In Step 503, a contact resistance between resistive materials in two electrodes is modulated by the contact pressure at the sensing point. In one or more embodiments, the resistive materials in the two electrodes are brought into contact by the contact pressure where the contact resistance is substantially a linear function of the contact pressure within the linear sensitivity range. In contrast, the contact resistance may be substantially independent of the contact pressure when the contact pressure is outside of the linear sensitivity range. For example, the contact resistance may approach infinity when the contact pressure is less than the lower limit of the linear sensitivity range. In another example, the contact resistance may approach a constant when the contact pressure exceeds the upper limit of the linear sensitivity range.

In Step 504, in response to the contact pressure at each sensing point, an output signal is obtained using a sensing circuit of the sensor array. In one or more embodiments, the output signal is dependent on the contact resistance at the corresponding sensing point.

In Step 505, a value is calculated for each sensing point of the sensor array based on the corresponding output signal. In one or more embodiments, the values are aggregated across the 2D planar surface of the padding to generate topographical data that represents a weight distribution of the user on the surface of the padding.

In Step 506, a posture of the user and/or a movement by the user is determined based on a change in the weight distribution of the user. In one or more embodiments, the user is a patient and the posture of the user and/or the movement by the user is included in posture monitoring data to be displayed to a healthcare professional caring for the patient. From the user's posture and the movement by the user, healthcare professionals may determine changes in the user's breathing rhythm, the user's frequency of cough, and other actions of the user while the user is monitored using the sensor array.

In accordance with one or more embodiments, FIG. 6A shows an example circuit diagram of the sensor array (201) in the posture monitoring system (100), depicted in FIGS. 1, 2, and 3A-3C above, without false positive correction. In one or more embodiments, one or more of the modules and elements shown in FIG. 6A may be omitted, repeated, and/or substituted. Accordingly, embodiments of sensor pad for monitoring user posture should not be considered limited to the specific arrangements of modules shown in FIG. 6A.

As shown in FIG. 6A, the example circuit diagram of the sensor array (201) includes the first layer electrode (301), the second layer electrode (303), sensing points (601a-d), inputs (603a-c), and outputs (605a-d). Each of these components will be described below in more detail in connection with the descriptions of FIGS. 1, 2, and 3A-3C above.

In one or more embodiments, sensing points (601a-d) are the points of contact between the first layer electrode (301) and the second layer electrode (303). In particular, each sensing point corresponds to an opening site in the aforementioned spacer layer where an overlapping region (e.g., overlapping region (302) depicted in FIGS. 3A and 3B above) exists between the first layer electrode (301) and the second layer electrode (303) at the opening site. The number of sensing points of the sensor array (201) may vary based on the number of first layer electrode (301) and the second layer electrode (303) of the sensor array (201). In the example of FIG. 6A, there are a total of twelve sensing points including the sensing points (601a-d). In other words, the example circuit diagram depicted in FIG. 6A corresponds to a portion of the sensor array (201) depicted in FIG. 3A above.

In one or more embodiments, as the number of sensing points (e.g., sensing points (601a-d)) of the sensor array (201) increases, the resolution of the sensor array (201) also increases.

In one or more embodiments, the inputs (603a-c) are portions of the first layer electrode (301) that receive an input signal sent from the computing device (105) and the sensing circuit (103) through the wires (305) and connectors (307a-b). While six of the first layer electrode (301) are depicted in FIG. 3A, only a portion (i.e., three) of the first layer electrode (301) are depicted in FIG. 6A for illustration purpose. In one or more embodiments, the computing device (105) sends the input signal (613) via the sensing circuit (103) to each of the inputs (603a-c) on the first layer electrodes (301) in a sequential order (referred to as an input signal sequence) that may be repeated from time to time. In each of the repetitive input signal sequences, the input signal (613) includes an analog voltage pulse with a pre-determined pulse width (e.g., approximately 1 millisecond (ms)), repetition rate (width (e.g., approximately 1 pulse per 18 ms), and pulse voltage magnitude (e.g., approximately between 0 volt (V) and 3.3 V). The terms “input signal,” “analog voltage pulse,” and “analog voltage pulse of he input signal” may be used interchangeably depending on the context. The input signal sequences may repeat at a periodic rate, e.g., approximately 1.2 Hertz (hz). For example, each analog voltage pulse may include a direct current (DC) voltage magnitude and/or an alternating current (AC) voltage magnitude. In one or more embodiments, the computing device (105) sends the input signal as a digital pulse representing logic 1 or as a digital value of the DC or AC voltage magnitude. For example, the digital value may be a positive integer value representing the voltage magnitude such as in the set {0, 1, 2, 3, . . . , 255}. In response, the sensing circuit (103) converts the digital pulse or the digital value into the analog voltage pulse. As used herein, analog voltage pulse is a voltage pulse containing information represented by a continuous (i.e., analog) pulse voltage magnitude, which is in contrast to a digital pulse where information is represented by discrete (i.e., non-continuous) magnitudes of logic 1 or logic 0.

Only one of the inputs (603a-c) receives the analog voltage pulse from the computing device (105) at a given point in time within the input signal sequence. For example, when input (603a) receives the analog voltage pulse from the computing device (105), the remaining inputs (603b-c) do not receive the analog voltage pulse from the computing device (105) and are held at 0V.

In one or more embodiments, the outputs (605a-d) are portions of the second layer electrode (303) that transmit an output signal (615) to the computing device (105) through the wires (305), connectors (307a-b), and sensing circuit (103). While six of the second layer electrode (303) are depicted FIG. 3A, only a portion (i.e., four) of the second layer electrode (303) are depicted FIG. 6A for illustration purpose. In one or more embodiments, the computing device (105) monitors each of the outputs (605a-d) on the second layer electrodes (303) to detect any output signal (615) from the sensing circuit (103). In one or more embodiments, the output signal (615) includes an analog voltage pulse with a pulse width and repetition rate that are similar to the input signal (613), and an analog voltage magnitude as a fraction of the input signal (613). For example, each analog voltage pulse may include a DC voltage magnitude and/or an AC voltage magnitude. In one or more embodiments, each analog voltage pulse of the output signal (615) is generated by the sensor array (201) in response to a corresponding analog voltage pulse of the input signal (613). In one or more embodiments, the computing device (105) receives the output signal (615) as a digital value of the DC and/or AC voltage magnitude, which is converted from the analog voltage pulse by the sensing circuit (103).

In one or more embodiments, the number of inputs (603a-c) is directly determined by the number of first layer electrodes (301) in the sensor array (201), and that the number of outputs (605a-d) is directly determined by the number of second layer electrodes (303) in the sensor array (201). For example, as shown in FIG. 6A, the sensor array (201) is a three-by-four electrode layer array matrix that has three inputs (603a-c) and four outputs (605a-d).

In one or more embodiments, the output signals (615) generated at the outputs (605a-d) are dependent on the combination of which first layer electrode (301) is receiving the analog voltage pulse of the input signal (613) from the computing device (105) and which sensing points (601a-d) are pressed when the user (106) applies an external force to the surface of the sensor pad (101). The computing device (105) stores pulse voltage magnitudes of the received output signals (615) in a digital array matrix that is the same size as the electrode layer array matrix. In particular, each location of the digital array matrix corresponds to one of the sensing points (e.g., sensing points (601a-d)), i.e., an overlapping region of the first layer electrode (301) and the second layer electrode (303). If the computing device (105) sends the input signal (613) to a particular first layer electrode (301) and receives, at substantially the same time, an analog voltage pulse of the output signal (615) at a particular second layer electrode (303), the computing device (105) stores the pulse voltage magnitude of the received output signal (615) in a location in the digital array matrix that corresponds to the sensing point where the contact resistance is reduced due to an external force is applied by the user. In one or more embodiments, the pulse voltage magnitude of the output signal (615) stored in each location in the digital array matrix represents a measure of the external force exerted at a corresponding sensing point. As the computing device (105) scans the sensor array (201) through one or more input signal sequences, the resultant contents of the digital array matrix correspond to topographical data, such as the topographical data depicted in FIG. 4 above.

In one or more embodiments, when the input signal (613) is applied to the input (603a) and the user (106) applies an external force on the sensing point (601a), the output (605a) generates an analog voltage pulse of the output signal (615) because the first layer electrode (301) is in electrical contact with the second layer electrode (303) at the sensing point (601a). For example, as shown in further details in FIG. 6C below, the input signal (613) applied to the sensing point (601a) generates the output signal (615) with a reduced pulse voltage magnitude based on a voltage divider circuit of a bias resistance and the contact resistance at the sensing point (601a). As noted above, the contact resistance is substantially a linear function of the contact pressure at the sensing point (601a) and, therefore, the pulse voltage magnitude of the output signal (615) represents the contact pressure at the sensing point (601a). For example, the output signal (615) is converted by the sensing circuit (103) into a digital value representing the pulse voltage magnitude of the output signal (615) as well as representing the magnitude of the contact pressure. The magnitude of the contact pressure at the sensing point (601a) is stored in a location designated for the sensing point (601a) in the digital array matrix of the computing device (105). This output signal (615) generated at the output (605a) when only the sensing point (601a) is pressed by the user (106) is a correct signal output.

In one or more embodiments, the sensor array (201) may also output an incorrect output signal. The incorrect output signal is a signal that returns a false positive result where a sensing point (601a-d) that is not pressed by the user (106) is detected as being pressed. For example, in the situation where the sensing points (601b-d) are simultaneously pressed by the user (106) and the sensing point (601a) is not pressed, the analog voltage pulse applied to the input (603a) may cause an analog current (referred to as a ghost current) to flow through the sensing point (601c), sensing point (601d), and sensing point (601b) to the output (605a) as if an analog voltage pulse is generated based on the contact resistance of the sensing point (601a) even though the sensing point (601a) is not pressed by the user (106).

In accordance with one or more embodiments, FIG. 6B shows an example circuit diagram of the sensor array (201) in the posture monitoring system (100), depicted in FIGS. 1, 2, and 3A-3C above, with false positive correction. In one or more embodiments, one or more of the modules and elements shown in FIG. 6B may be omitted, repeated, and/or substituted. Accordingly, embodiments of sensor pad for monitoring user posture should not be considered limited to the specific arrangements of modules shown in FIG. 6B.

As shown in FIG. 6B, the example circuit diagram of the sensor array (201) is based on the example circuit diagram of the sensor array (201) depicted in FIG. 6A above with the addition of second layer electrode inputs (701a-d), which will be described below in more detail in connection with the descriptions of FIGS. 1, 2, and 3A-3C above.

In one or more embodiments, the second layer electrode inputs (701a-d) are portions of the second layer electrode (303) that receive a de-ghosting signal (711) from the sensor correction circuit (109), depicted in FIG. 1 above, that is controlled by the computing device (105) through the wires (305) and an additional connector (307). As discussed in further detail in reference to FIG. 3C below, the de-ghosting signal (711) is a signal for preventing or otherwise suppressing the ghost current from flowing through the sensor array (201).

In one or more embodiments, the number of second layer electrode inputs (701a-d) is directly determined by the number of second layer electrodes (303) in the sensor array (201). For example, As shown in FIG. 6B, the sensor array (201) has four second layer electrodes (303) and four second layer electrode inputs (701a-d).

The computing device (105) controls the sensor correction circuit (109), depicted in FIG. 1 above, to send the de-ghosting signal (711) to each of the second layer electrode inputs (701a-d). In one or more embodiments, the computing device (105) sends the de-ghosting signal (711) as a digital pulse representing logic 1 or a digital value representing a DC or AC magnitude of an analog pulse. In response, the sensor correction circuit (109) converts the digital pulse or digital value into a corresponding analog voltage pulse for sending to each of the second layer electrode inputs (701a-d).

In one or more embodiments, if the input (603a) (i.e., input being scanned) receives the input signal (613) from the sensing circuit (103) to detect whether the sensing point (601a) is being pressed, the second layer electrode input (701a) does not receive the de-ghosting signal (711) from the sensor correction circuit (109) or the computing device (105). In contrast, each of the remaining second layer electrode inputs (701c-d) (i.e., inputs not being scanned) receives the de-ghosting signal (711) from the sensor correction circuit (109) to prevent or otherwise suppress the ghost current e.g., flowing through the sensing point (601c) and sensing point (601d) when the sensing points (601b-d) are simultaneously pressed.

According to the foregoing, the sensing circuit (103) is coupled to the first layer electrodes (301) via the inputs (603a-c) to send an input signal (613) to a single first layer electrode (e.g., via input (603a)) at a particular point in time, where a remainder portion of the first layer electrodes (301) (e.g., at inputs (603b-c)) is prevented from receiving the input signal (613) at the particular point in time. In addition, the sensing circuit (103) is also coupled to the second layer electrodes (303) via the outputs (605a-d) to detect, at the particular point in time, an output signal (615) from a second layer electrode (e.g., via output (605a)), where a remainder portion of the second layer electrodes (303) (e.g., at outputs (605b-d)) is temporarily ignored. In particular, the combination of the single first layer electrode and the second layer electrode corresponds to a sensing point where contact resistance is reduced due to an external force.

Further, the sensor correction circuit (109) is coupled to the second layer electrodes (303) to send, at the particular point in time, a de-ghosting signal (711) to the aforementioned remainder portion of the second layer electrodes (303) (e.g., via second layer inputs (7016b-d)), where the second layer electrode (e.g., at second layer input (701a)) is prevented from receiving the de-ghosting signal (711) at the particular point in time. Further details of the sensor correction circuit (109) are described in reference to FIG. 6C below.

In accordance with one or more embodiments, FIG. 6C shows an example schematic circuit diagram of the posture monitoring system (100), depicted in FIG. 1 above. In one or more embodiments, one or more of the modules and elements shown in FIG. 6C may be omitted, repeated, and/or substituted. Accordingly, embodiments of sensor pad for monitoring user posture should not be considered limited to the specific arrangements of modules shown in FIG. 6C.

As shown in FIG. 6C, the schematic circuit diagram of the posture monitoring system (100) includes the computing device (105), the sensing circuit (103), the sensor correction circuit (109), and a sensing point (601a) at the overlapping region (302). In particular, the sensing circuit (103) includes a demultiplexer circuit (103a) and an analog-to-digital (A/D) circuit (103b) that may be implemented using one or more discrete transistor circuitry and/or integrated circuit element. The demultiplexer circuit (103a) is configured to selectively apply the input signal (613) in the aforementioned input signal sequence to each input (603a), one at a time, of the first layer electrodes (301). The A/D circuit (103b) is configured to convert the analog voltage pulse of the output signal (615) into digital values representing the value of the variable contact resistance (611) when the user applies the external force to the overlapping region (302). For example, the magnitude of the input signal (613) is reduced by the voltage divider circuit, formed from the variable contact resistance (611) and the bias resistance (612), to generate the output signal (615). Accordingly, the reduced magnitude of the output signal (615) is proportional to the variable contact resistance (611), which in turn is inversely proportional to the contact pressure at the overlapping region (302). Accordingly, the reduced magnitude of the output signal (615) represents the external force applied by the user to the overlapping region (302).

At a particular point in time when a particular second layer electrode (303) (e.g., at output (605a)) is checked to detect any output signal (615) during the input signal sequence, the de-ghosting signal (711) is sent to the aforementioned remainder portion of the second layer electrodes (303) (e.g., via second layer inputs (701b-d)), where the particular second layer electrode being checked is prevented from receiving (e.g., at the second layer input (701a)) the de-ghosting signal (711) at the particular point in time. For example, each of the second layer inputs (701a-d) may be driven by an open-collector (OC) circuit (109a) of the sensor collection circuit (109) so as to avoid interfering with the aforementioned voltage divider circuit when not receiving the de-ghosting signal (711). The OC circuit (109a) may be implemented using one or more discrete transistor circuitry and/or integrated circuit element In particular, when the second layer inputs (701a) is not receiving the de-ghosting signal (711), the output node of the OC circuit (109a) presents negligible impedance loading to the voltage divider circuit, formed from the variable contact resistance (611) and the bias resistance (612). In contrast, when the output (605a) is not being checked for any output signal (615), the OC circuit (109a) presents the de-ghosting signal (711) to the voltage divider circuit via the second layer input (701a) such that the particular second layer electrode (303) connected to the output (605a) is clamped at a pre-determined voltage for suppressing the aforementioned ghost current.

Embodiments may be implemented on a computing system. Any combination of mobile, desktop, server, router, switch, embedded device, or other types of hardware may be used. For example, as shown in FIG. 7A, the computing system (700) may include one or more computer processors (702), non-persistent storage (704) (e.g., volatile memory, such as random access memory (RAM), cache memory), persistent storage (706) (e.g., a hard disk, an optical drive such as a compact disk (CD) drive or digital versatile disk (DVD) drive, a flash memory, etc.), a communication interface (712) (e.g., Bluetooth interface, infrared interface, network interface, optical interface, etc.), and numerous other elements and functionalities.

The computer processor(s) (702) may be an integrated circuit for processing instructions. For example, the computer processor(s) may be one or more cores or micro-cores of a processor. The computing system (700) may also include one or more input devices (710), such as a touchscreen, keyboard, mouse, microphone, touchpad, electronic pen, or any other type of input device.

The communication interface (712) may include an integrated circuit for connecting the computing system (700) to a network (not shown) (e.g., a local area network (LAN), a wide area network (WAN) such as the Internet, mobile network, or any other type of network) and/or to another device, such as another computing device.

Further, the computing system (700) may include one or more output devices (708), such as a screen (e.g., a liquid crystal display (LCD), a plasma display, touchscreen, cathode ray tube (CRT) monitor, projector, or other display device), a printer, external storage, or any other output device. One or more of the output devices may be the same or different from the input device(s). The input and output device(s) may be locally or remotely connected to the computer processor(s) (702), non-persistent storage (704), and persistent storage (706). Many different types of computing systems exist, and the aforementioned input and output device(s) may take other forms.

Software instructions in the form of computer readable program code to perform embodiments of the invention may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the invention.

The computing system (700) in FIG. 7A may be connected to or be a part of a network. For example, as shown in FIG. 7B, the network (720) may include multiple nodes (e.g., node X (722), node Y (724)). Each node may correspond to a computing system, such as the computing system shown in FIG. 7A, or a group of nodes combined may correspond to the computing system shown in FIG. 7A. By way of an example, embodiments of the invention may be implemented on a node of a distributed system that is connected to other nodes. By way of another example, embodiments of the invention may be implemented on a distributed computing system having multiple nodes, where each portion of the invention may be located on a different node within the distributed computing system. Further, one or more elements of the aforementioned computing system (700) may be located at a remote location and connected to the other elements over a network.

Although not shown in FIG. 7B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (722), node Y (724)) in the network (720) may be configured to provide services for a client device (726). For example, the nodes may be part of a cloud computing system. The nodes may include functionality to receive requests from the client device (726) and transmit responses to the client device (726). The client device (726) may be a computing system, such as the computing system shown in FIG. 7A. Further, the client device (726) may include and/or perform all or a portion of one or more embodiments of the invention.

The computing system or group of computing systems described in FIGS. 7A and 7B may include functionality to perform a variety of operations disclosed herein. For example, the computing system(s) may perform communication between processes on the same or different system. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file. Further details pertaining to a couple of these non-limiting examples are provided below.

Based on the client-server networking model, sockets may serve as interfaces or communication channel end-points enabling bidirectional data transfer between processes on the same device. Foremost, following the client-server networking model, a server process (e.g., a process that provides data) may create a first socket object. Next, the server process binds the first socket object, thereby associating the first socket object with a unique name and/or address. After creating and binding the first socket object, the server process then waits and listens for incoming connection requests from one or more client processes (e.g., processes that seek data). At this point, when a client process wishes to obtain data from a server process, the client process starts by creating a second socket object. The client process then proceeds to generate a connection request that includes at least the second socket object and the unique name and/or address associated with the first socket object. The client process then transmits the connection request to the server process. Depending on availability, the server process may accept the connection request, establishing a communication channel with the client process, or the server process, busy in handling other operations, may queue the connection request in a buffer until server process is ready. An established connection informs the client process that communications may commence. In response, the client process may generate a data request specifying the data that the client process wishes to obtain. The data request is subsequently transmitted to the server process. Upon receiving the data request, the server process analyzes the request and gathers the requested data. Finally, the server process then generates a reply including at least the requested data and transmits the reply to the client process. The data may be transferred, more commonly, as datagrams or a stream of characters (e.g., bytes).

Shared memory refers to the allocation of virtual memory space in order to substantiate a mechanism for which data may be communicated and/or accessed by multiple processes. In implementing shared memory, an initializing process first creates a shareable segment in persistent or non-persistent storage. Post creation, the initializing process then mounts the shareable segment, subsequently mapping the shareable segment into the address space associated with the initializing process. Following the mounting, the initializing process proceeds to identify and grant access permission to one or more authorized processes that may also write and read data to and from the shareable segment. Changes made to the data in the shareable segment by one process may immediately affect other processes, which are also linked to the shareable segment. Further, when one of the authorized processes accesses the shareable segment, the shareable segment maps to the address space of that authorized process. Often, only one authorized process may mount the shareable segment, other than the initializing process, at any given time.

Other techniques may be used to share data, such as the various data described in the present application, between processes without departing from the scope of the invention. The processes may be part of the same or different application and may execute on the same or different computing system.

Rather than or in addition to sharing data between processes, the computing system performing one or more embodiments of the invention may include functionality to receive data from a user. For example, in one or more embodiments, a user may submit data via a graphical user interface (GUI) on the user device. Data may be submitted via the graphical user interface by a user selecting one or more graphical user interface widgets or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

By way of another example, a request to obtain data regarding the particular item may be sent to a server operatively connected to the user device through a network. For example, the user may select a uniform resource locator (URL) link within a web client of the user device, thereby initiating a Hypertext Transfer Protocol (HTTP) or other protocol request being sent to the network host associated with the URL. In response to the request, the server may extract the data regarding the particular selected item and send the data to the device that initiated the request. Once the user device has received the data regarding the particular item, the contents of the received data regarding the particular item may be displayed on the user device in response to the user's selection. Further to the above example, the data received from the server after selecting the URL link may provide a web page in Hyper Text Markup Language (HTML) that may be rendered by the web client and displayed on the user device.

Once data is obtained, such as by using techniques described above or from storage, the computing system, in performing one or more embodiments of the invention, may extract one or more data items from the obtained data. For example, the extraction may be performed as follows by the computing system in FIG. 7A. First, the organizing pattern (e.g., grammar, schema, layout) of the data is determined, which may be based on one or more of the following: position (e.g., bit or column position, Nth token in a data stream, etc.), attribute (where the attribute is associated with one or more values), or a hierarchical/tree structure (consisting of layers of nodes at different levels of detail-such as in nested packet headers or nested document sections). Then, the raw, unprocessed stream of data symbols is parsed, in the context of the organizing pattern, into a stream (or layered structure) of tokens (where each token may have an associated token “type”).

Next, extraction criteria are used to extract one or more data items from the token stream or structure, where the extraction criteria are processed according to the organizing pattern to extract one or more tokens (or nodes from a layered structure). For position-based data, the token(s) at the position(s) identified by the extraction criteria are extracted. For attribute/value-based data, the token(s) and/or node(s) associated with the attribute(s) satisfying the extraction criteria are extracted. For hierarchical/layered data, the token(s) associated with the node(s) matching the extraction criteria are extracted. The extraction criteria may be as simple as an identifier string or may be a query presented to a structured data repository (where the data repository may be organized according to a database schema or data format, such as XML).

The extracted data may be used for further processing by the computing system. For example, the computing system of FIG. 7A, while performing one or more embodiments of the invention, may perform data comparison. Data comparison may be used to compare two or more data values (e.g., A, B). For example, one or more embodiments may determine whether A>B, A=B, A !=B, A<B, etc. The comparison may be performed by submitting A, B, and an opcode specifying an operation related to the comparison into an arithmetic logic unit (ALU) (i.e., circuitry that performs arithmetic and/or bitwise logical operations on the two data values). The ALU outputs the numerical result of the operation and/or one or more status flags related to the numerical result. For example, the status flags may indicate whether the numerical result is a positive number, a negative number, zero, etc. By selecting the proper opcode and then reading the numerical results and/or status flags, the comparison may be executed. For example, in order to determine if A>B, B may be subtracted from A (i.e., A−B), and the status flags may be read to determine if the result is positive (i.e., if A>B, then A−B>0). In one or more embodiments, B may be considered a threshold, and A is deemed to satisfy the threshold if A=B or if A>B, as determined using the ALU. In one or more embodiments of the invention, A and B may be vectors, and comparing A with B requires comparing the first element of vector A with the first element of vector B, the second element of vector A with the second element of vector B, etc. In one or more embodiments, if A and B are strings, the binary values of the strings may be compared.

The computing system in FIG. 7A may implement and/or be connected to a data repository. For example, one type of data repository is a database. A database is a collection of information configured for ease of data retrieval, modification, re-organization, and deletion. Database Management System (DBMS) is a software application that provides an interface for users to define, create, query, update, or administer databases.

The user, or software application, may submit a statement or query into the DBMS. Then the DBMS interprets the statement. The statement may be a select statement to request information, update statement, create statement, delete statement, etc. Moreover, the statement may include parameters that specify data, or data container (database, table, record, column, view, etc.), identifier(s), conditions (comparison operators), functions (e.g. join, full join, count, average, etc.), sort (e.g. ascending, descending), or others. The DBMS may execute the statement. For example, the DBMS may access a memory buffer, a reference or index a file for read, write, deletion, or any combination thereof, for responding to the statement. The DBMS may load the data from persistent or non-persistent storage and perform computations to respond to the query. The DBMS may return the result(s) to the user or software application.

The computing system of FIG. 7A may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a data model.

For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data may also be presented through various audio methods. In particular, data may be rendered into an audio format and presented as sound through one or more speakers operably connected to a computing device.

Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The above description of functions presents only a few examples of functions performed by the computing system of FIG. 7A and the nodes and/or client device in FIG. 7B. Other functions may be performed using one or more embodiments of the invention.

While one or more embodiments have been described with respect to a limited number of embodiments, those skilled in the art, having benefit of this disclosure, will appreciate that other embodiments may be devised which do not depart from the scope as disclosed herein. Accordingly, the scope should be limited only by the attached claims.

What is claimed is:

1. A sensor pad for monitoring user posture, comprising:
a sensor array comprising:
a plurality of first layer electrodes disposed on a first substrate that faces a second substrate;
a plurality of second layer electrodes disposed on a second substrate that faces the first substrate;
a spacer layer disposed between the first substrate and the second substrate and comprising a plurality of holes each corresponding to an overlapping region of the plurality of first layer electrodes and the plurality of second layer electrodes, wherein the plurality of first layer electrodes are configured to contact the plurality of second layer electrodes through at least one of the plurality of holes via a corresponding overlapping region when an external force is applied on the sensor pad by a user; and
a sensing circuit configured to generate an output signal in response to the external force.

2. The sensor pad of claim 1, further comprising:
a padding disposed between the sensor array and the user, wherein the padding is configured to transfer, collectively with the first substrate and the second substrate, the external force into a contact pressure in the corresponding overlapping region of the at least one of the plurality of holes.

3. The sensor pad of claim 1,
wherein the plurality of first layer electrodes comprise a first electrically resistive material,
wherein the plurality of second layer electrodes comprise a second electrically resistive material, and
wherein the output signal is based on a contract resistance, between the first electrically resistive material and the second electrically resistive material, in the corresponding overlapping region of the at least one of the plurality of holes.

4. The sensor pad of claim 2,
wherein the external force comprises a weight of the user on a surface of the sensor pad,
wherein the padding is configured to maintain the contact pressure within a pre-determined range based on the weight of the user, and
wherein a contact resistance is substantially proportional to the contact pressure within the pre-determined range.

5. The sensor pad of claim 1, further comprising:
a computing device coupled to the sensing circuit and configured to calculate a value based on the output signal and generate topographical data of a surface of the sensor pad based at least on the value,
wherein the topographical data represents a weight distribution of the user on the surface of the sensor pad.

6. The sensor pad of claim 1, further comprising:
a sensor correction circuit configured to prevent the sensing circuit from generating a false positive signal in the output signal.

7. The sensor pad of claim 6,
wherein the sensing circuit is coupled to the plurality of first layer electrodes to send an input signal to a single first layer electrode of the plurality of first electrodes at a particular point in time, wherein a first remainder portion of the plurality of first layer electrodes excluding said single first layer electrode is prevented from receiving the input signal at the particular point in time,
wherein the sensing circuit is further coupled to the plurality of second layer electrodes to detect, at the particular point in time, an output signal from a second layer electrode of the plurality of second layer electrodes, wherein a second remainder portion of the plurality of second layer electrodes excluding said second layer electrode is temporarily ignored, wherein the topographical data is based on a combination of the single first layer electrode and the second layer electrode, and
wherein the sensor correction circuit is coupled to the plurality of second layer electrodes to send, at the particular point in time, a de-ghosting signal to the second remainder portion of the plurality of second layer electrodes excluding said second layer electrode, wherein said second layer electrode is prevented from receiving the de-ghosting signal at the particular point in time.

8. The sensor pad of claim 5, wherein the computing device is further configured to determine at least one selected from a group consisting of a posture of the user and a movement by the user based on a change in the weight distribution of the user.

9. The sensor pad of claim 1, wherein the sensor pad is comprised in at least one selected from a group consisting of a mattress, a seat cushion, a shoe pad, and a wearable article worn by the user.

10. A system for monitoring user posture, comprising:
a display configured to display posture monitoring data of a user;
a sensor array comprising:
a plurality of first layer electrodes disposed on a first substrate that faces a second substrate;
a plurality of second layer electrodes disposed on a second substrate that faces the first substrate;
a spacer layer disposed between the first substrate and the second substrate and comprising a plurality of holes each corresponding to an overlapping region of the plurality of first layer electrodes and the plurality of second layer electrodes, wherein the plurality of first layer electrodes are configured to contact the plurality of second layer electrodes through at least one of the plurality of holes via a corresponding overlapping region when an external force is applied on the padding by the user; and
a sensing circuit configured to generate an output signal in response to the external force,
wherein the display posture monitoring data is based at least on the output signal.

11. The system of claim 10, further comprising:
a padding disposed between the sensor array and the user, wherein the padding is configured to transfer, collectively with the first substrate and the second substrate, the external force into a contact pressure in the corresponding overlapping region of the at least one of the plurality of holes.

12. The system of claim 10,
wherein the plurality of first layer electrodes comprise a first electrically resistive material,
wherein the plurality of second layer electrodes comprise a second electrically resistive material, and
wherein the output signal is based on a contract resistance, between the first electrically resistive material and the second electrically resistive material, in the corresponding overlapping region of the at least one of the plurality of holes.

13. The system of claim 11,
wherein the external force comprises a weight of the user on a surface of the padding,
wherein the padding is configured to maintain the contact pressure within a pre-determined range based on the weight of the user, and
wherein a contact resistance is substantially proportional to the contact pressure within the pre-determined range.

14. The system of claim 10, further comprising:
a computing device coupled to the sensing circuit and configured to calculate a value based on the output signal and generate topographical data of a surface of the padding based at least on the value,
wherein the topographical data represents a weight distribution of the user on the surface of the padding, and
wherein the posture monitoring data comprises the topographical data.

15. The system of claim 10, further comprising:
a sensor correction circuit configured to prevent the sensing circuit from generating a false positive signal in the output signal.

16. The system of claim 15,
wherein the sensing circuit is coupled to the plurality of first layer electrodes to send an input signal to a single first layer electrode of the plurality of first electrodes at a particular point in time, wherein a first remainder portion of the plurality of first layer electrodes excluding said single first layer electrode is prevented from receiving the input signal at the particular point in time,
wherein the sensing circuit is further coupled to the plurality of second layer electrodes to detect, at the particular point in time, an output signal from a second layer electrode of the plurality of second layer electrodes, wherein a second remainder portion of the plurality of second layer electrodes excluding said second layer electrode is temporarily ignored, wherein the topographical data is based on a combination of the single first layer electrode and the second layer electrode, and
wherein the sensor correction circuit is coupled to the plurality of second layer electrodes to send, at the particular point in time, a de-ghosting signal to the second remainder portion of the plurality of second layer electrodes excluding said second layer electrode, wherein said second layer electrode is prevented from receiving the de-ghosting signal at the particular point in time.

17. The system of claim 14,
wherein the computing device is further configured to determine, based on a change in the weight distribution of the user, at least one selected from a group consisting of a posture of the user and a movement by the user, and
wherein the posture monitoring data comprises at least one selected from a group consisting of the posture of the user and the movement by the user.

18. The system of claim 11, wherein the sensor array and the padding are comprised in at least one selected from a group consisting of a mattress, a seat cushion, a shoe pad, and a wearable article worn by the user.

19. A method for monitoring user posture, comprising:
disposing a sensor array under a user, the sensor array comprising:
a plurality of first layer electrodes disposed on a first substrate that faces a second substrate;
a plurality of second layer electrodes disposed on a second substrate that faces the first substrate; and
a spacer layer disposed between the first substrate and the second substrate and comprising a plurality of holes each corresponding to an overlapping region of the plurality of first layer electrodes and the plurality of second layer electrodes,
wherein the plurality of first layer electrodes are configured to contact the plurality of second layer electrodes through at least one of the plurality of holes via a corresponding overlapping region when an external force is applied on the sensor array by a user;
obtaining, using a sensing circuit of the sensor array, an output signal in response to the external force; and
displaying, based on the output signal, posture monitoring data of the user.

20. The method of claim 19,
wherein the plurality of first layer electrodes comprise a first electrically resistive material,
wherein the plurality of second layer electrodes comprise a second electrically resistive material, and
wherein the output signal is based on a contract resistance, between the first electrically resistive material and the second electrically resistive material, in the corresponding overlapping region of the at least one of the plurality of holes.

21. The method of claim 20, further comprising:
disposing a padding between the sensor array and the user, wherein the padding is configured to transfer, collectively with the first substrate and the second substrate, the external force into a contact pressure in the corresponding overlapping region of the at least one of the plurality of holes;
wherein the external force comprises a weight of the user on a surface of the padding,
wherein the padding is configured to maintain the contact pressure within a pre-determined range based on the weight of the user, and
wherein the contact resistance is substantially proportional to the contact pressure within the pre-determined range.

22. The method of claim 19, further comprising:
calculating a value based on the output signal;
generating topographical data of a surface of the padding based at least on the value,
wherein the topographical data represents a weight distribution of the user on the surface of the padding.

23. The method of claim 19, further comprising:
preventing, using a sensor correction circuit, the sensing circuit from generating a false positive signal in the output signal.

24. The method of claim 19, further comprising:
determining, based on a change in the weight distribution of the user, at least one selected from a group consisting of a posture of the user and a movement by the user, and
wherein the posture monitoring data comprises at least one selected from a group consisting of the posture of the user and the movement by the user.
